(19) United States
(12) Patent Application Publication
(10) Pub. No.: US 2019/0029503 A1
(43) Pub. Date: Jan. 31, 2019

(54) CHANNEL TUBE FOR ENDOSCOPE

(71) Applicant: OLYMPUS CORPORATION, Tokyo (JP)

(72) Inventors: Takaaki HANAI, Sagamihara-shi (JP); Kiyoshi TAKAO, Sagamihara-shi (JP); Yoshiteru NONAKA, Yokohama-shi (JP); Yoshinori HIGUCHI, Tokyo (JP)

(73) Assignee: OLYMPUS CORPORATION, Tokyo (JP)

(21) Appl. No.: 16/151,149

(22) Filed: Oct. 3, 2018

Related U.S. Application Data
(63) Continuation of application No. PCT/JP2017/013922, filed on Apr. 3, 2017.

(30) Foreign Application Priority Data
Apr. 7, 2016 (JP) 2016-077251

(51) Int. Cl. A61B 1/018; A61B 1/00; A61M 25/00

(52) U.S. Cl. CPC A61B 1/018; A61M 25/005; A61B 1/00064

(57) ABSTRACT

The channel tube for endoscope according to the present invention is provided with: an inner-layer tube inside which a through hole extending in a longitudinal direction is formed, the inner-layer tube having as a base material thereof an elastomer or a flexible resin; an elastomer layer comprising a polymer elastomer, the elastomer layer being disposed so as to cover the outside of the inner-layer tube, and the surface of the elastomer layer being exposed to the outside; a reinforcing layer part including a flexible reinforcing member, the reinforcing layer part being disposed so as to surround the inner-layer tube; and a buffer layer part which deforms more readily than the reinforcing layer part, the buffer layer part being layered on the reinforcing layer part and disposed between the inner-layer tube and an outer peripheral surface of the elastomer layer.

### CROSS REFERENCE TO RELATED APPLICATIONS

This application is a continuation application based on a PCT Patent Application No. PCT/JP2017/013922, filed on Apr. 3, 2017, whose priority is claimed on Japanese Patent Application No. 2016-077251, filed on Apr. 7, 2016, the entire contents of which are hereby incorporated by reference.

### BACKGROUND

### Technical Field

The present invention relates to a channel tube for endoscope.

### Background Art

In recent years, compatibility between flexibility and kink resistance is required in a channel tube for endoscope.

For example, in the treatment instrument insertion channel described in Japanese Unexamined Patent Application, First Publication No. Hei 3-205022, a net made of a stainless steel wire is covered on a tube main body made of urethane resin having an inner surface coating layer of Teflon (registered trademark) formed on the inner surface. The metallic net expands and contracts easily when being bent, thereby resistance against bending is small, and it has also shape retentivity, it has flexibility and kink resistance.

A tube for endoscope described in Japanese Unexamined Patent Application, First Publication No. 2010-29435 includes a tube main body made of a fluororesin, a reinforcing tape wound and fixed around the outer circumferential surface of the tube main body, and a polyurethane outer covering that covers the tube main body from above the reinforcing tape. In the reinforcing tape, anisotropy is imparted to the rigidity in the axial direction and the circumferential direction by forming the strand of polyester resin in a net-like shape. With the reinforcing tape, the endoscope tube has flexibility and kink resistance.

### SUMMARY

A channel tube for endoscope includes: an inner-layer tube inside which a through hole extending in a longitudinal direction is formed, the inner-layer tube having an elastomer or a flexible resin as a base material thereof; an elastomer layer including a polymer elastomer, the elastomer layer being disposed so as to cover an outside of the inner-layer tube, and the surface of the elastomer layer being exposed to an outside; a reinforcing layer part including a flexible reinforcing member, the reinforcing layer part being disposed so as to surround the inner-layer tube; and a buffer layer part that deforms more readily than the reinforcing layer part, the buffer layer part being layered on the reinforcing layer part and disposed between the inner-layer tube and an outer peripheral surface of the elastomer layer.

The reinforcing member may include a first mesh-like body formed of a first element wire.

The buffer layer part may include a second mesh-like body formed of a second element wire that is softer than the first element wire.

The buffer layer part may be disposed between the inner-layer tube and the reinforcing layer part.

The buffer layer part may be disposed between the reinforcing layer part and an outer peripheral surface of the elastomer layer.

The buffer layer part may include: an inner buffer layer part disposed between the inner-layer tube and the reinforcing layer part; and an outer buffer layer part disposed between the reinforcing layer part and an outer peripheral surface of the elastomer layer.

The inner-layer tube may be made of a fluororesin.

The elastomer layer may be disposed so as to penetrate through the reinforcing layer part and the buffer layer part so as to be in close contact with an outer peripheral surface of the inner-layer tube, and the elastomer layer may be formed of an elastomer having a lower adhesion to the first mesh-like body and the second mesh-like body than an adhesion to the inner-layer tube.

The elastomer may include an organic peroxide crosslinked rubber or a thermoplastic elastomer in which the organic peroxide crosslinked rubber is dispersed.

At least one of the first mesh-like body and the second mesh-like body may be partly exposed to an outside from the outer peripheral surface.

### BRIEF DESCRIPTION OF THE DRAWINGS

FIG. 1 is a schematic partial sectional view showing a configuration example of a channel tube for endoscope according to a first embodiment of the present invention.

FIG. 2 is a schematic cross-sectional view showing a configuration example of a channel tube for endoscope of a modified example (first modified example) of the first embodiment of the present invention.

FIG. 3 is a schematic partial sectional view showing a configuration example of a channel tube for endoscope according to a second embodiment of the present invention.

FIG. 4 is a schematic cross-sectional view showing a configuration example of a channel tube for endoscope of a modified example (second modified example) of the second embodiment of the present invention.

FIG. 5 is a schematic partial sectional view showing a configuration example of a channel tube for endoscope according to a third embodiment of the present invention.

FIG. 6 is a schematic cross-sectional view showing a configuration example of a channel tube for endoscope of a modified example (third modified example) of the third embodiment of the present invention.

FIG. 7 is a schematic partial sectional view showing a configuration example of a channel tube for endoscope according to a fourth embodiment of the present invention.

FIG. 8 is a schematic partial sectional view showing a configuration of a channel tube for endoscope of Comparative Example 1.

### DETAILED DESCRIPTION OF THE PREFERRED EMBODIMENTS

Embodiments of the present invention will be described below with reference to the accompanying drawings. In all drawings, the same or corresponding members are denoted by the same reference numerals, and description common to them is omitted even if the embodiment is different.

### First Embodiment

A channel tube for endoscope according to a first embodiment of the present invention will be described.

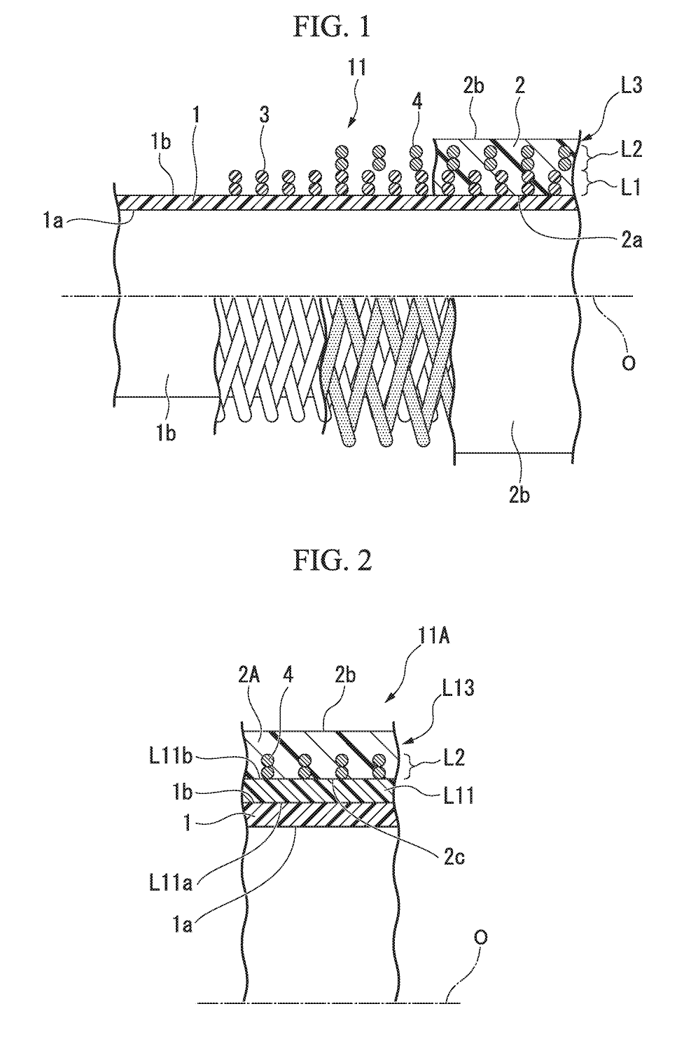

FIG. 1 is a schematic partial sectional view showing a configuration example of a channel tube for endoscope according to a first embodiment of the present invention.

As shown in FIG. 1, the channel tube for endoscope 11 of the present embodiment includes an inner-layer tube 1, a buffer layer part L1 (inner buffer layer part), a reinforcing layer part L2, and an outer layer part L3.

In the endoscope apparatus, the channel tube for endoscope 11 is used, for example, as a treatment instrument channel through which a treatment instrument etc. is inserted.

The inner-layer tube 1 has a through hole extending in the longitudinal direction inside thereof and is a tubular member made of an elastomer or a resin having flexibility as a base material. A shaft-like or tubular insertion member such as a treatment tool, a catheter, or the like, for example, can be inserted into the inner side of the inner peripheral surface 1a of the inner-layer tube 1 where the through hole is formed.

As the material of the base material of the inner-layer tube 1, general-purpose plastics such as, for example, polyethylene, polypropylene, polystyrene, polyvinyl chloride, polymethyl methacrylate, polymethyl acrylate, acrylonitrile-butadiene-styrene, acrylonitrile-styrene, polyvinyl alcohol, polyester, polyethylene terephthalate, polyurethane, polymethylpentene, brominated polyethylene, ethylene-vinyl acetate copolymer, ethylene-vinyl alcohol copolymer, ethylene-methyl acrylate copolymer, ionomer, or the like may be used.

As the material of the base material of the inner-layer tube 1, engineering plastic such as, for example, polycarbonate, polyacetal, polyamide, polybutylene terephthalate, polybutylene naphthalate, polyethylene naphthalate, or the like may be used.

As the material of the base material of the inner-layer tube 1, super engineering plastics such as, for example, polyphenylene sulfide, polyether imide, polysulfone, polyarylate, polyimide, polyether sulfone, polyamide imide, polyether ether ketone, polyallyl ether ketone, polyether nitrile, or the like may be used.

As the material of the base material of the inner-layer tube 1, fluorine resin such as, for example, polytetrafluoroethylene, ethylene-tetrafluoroethylene copolymer, tetrafluoroethylene-hexafluoropropylene copolymer, polychlorotrifluoroethylene, tetrafluoroethylene-perfluoro alkyl vinyl ether copolymer, polyvinylidene fluoride, chlorotrifluoroethylene-ethylene copolymer, or the like may be used.

As the material of the base material of the inner-layer tube 1, a thermoplastic elastomer such as, for example, urethane type thermoplastic elastomers, ester type thermoplastic elastomers, amide type thermoplastic elastomers, styrene type thermoplastic elastomers, olefin type thermoplastic elastomers, fluorine type thermoplastic elastomers, a vinyl based thermoplastic elastomer, or the like may be used.

Each of the above-described materials may be used alone for the inner-layer tube 1, or may be used as a composite material in which a plurality of materials are combined.

Among the above-mentioned materials, the inner-layer tube 1 is more preferably made of a fluororesin because it is excellent in chemical resistance to chemicals used for sterilization treatment and the like. Among fluororesins, for example, polytetrafluoroethylene and tetrafluoroethylene-perfluoroalkyl vinyl ether copolymer are excellent in chemical resistance. Among these, polytetrafluoroethylene is particularly preferable because it is particularly excellent in chemical resistance.

The inner peripheral surface 1a of the inner-layer tube 1 is repeatedly cleaned. Therefore, from the viewpoint of ease of cleaning, the inner peripheral surface 1a is more preferably a smooth surface. If the inner peripheral surface 1a is a smooth surface, the treatment instrument and the like inserted into the inner peripheral surface 1a also slide smoothly.

In order to make the inner peripheral surface 1a a smooth surface, at least the portion exposed as the inner peripheral surface 1a may be made of a non-porous material.

The inner peripheral surface 1a of the inner-layer tube 1 may be formed of a coating resin coated on the base material.

Examples of the coating resin capable of forming the inner peripheral surface 1a of the inner-layer tube 1 are, for example, polytetrafluoroethylene, ethylene-tetrafluoroethylene copolymer, tetrafluoroethylene-hexafluoropropylene copolymer, polychloro trifluoroethylene, tetrafluoroethylene-perfluoroalkyl vinyl ether copolymer, polyvinylidene fluoride, chlorotrifluoroethylene-ethylene copolymer, and the like.

By forming the inner peripheral surface 1a with these coating resins, the slipperiness on the inner peripheral surface 1a can be improved.

As the base material of the inner-layer tube 1, the above-mentioned base material or a porous material or foam material of a composite material may be used. In this case, the flexibility of the inner-layer tube 1 is improved.

The inner peripheral surface 2a of the elastomer layer 2 to be described later is brought into close contact with the outer peripheral surface 1b of the inner-layer tube 1. For this reason, the outer peripheral surface 1b may be subjected to a surface treatment for improving adhesion as necessary.

Examples of the surface treatment method include a chemical etching treatment with a metallic sodium solution, a treatment by plasma irradiation, a polishing treatment by machining, and the like.

The inner-layer tube 1 is required to have chemical resistance, biocompatibility, washing and disinfecting property, airtightness, and liquid tightness. From the viewpoint of particularly satisfying these characteristics, it is more preferable that a non-porous fluororesin is used as the material of the inner-layer tube 1.

Since the fluororesin is also excellent in slipperiness, the frictional force with a hard member such as a treatment tool is reduced, whereby the kink resistance is further improved because the abrasion amount is reduced.

The buffer layer part L1, the reinforcing layer part L2, and the outer layer part L3 are tubular layered portions surrounding the outer peripheral surface 1b outside the outer peripheral surface 1b of the inner-layer tube 1. The buffer layer part L1, the reinforcing layer part L2, and the outer layer part L3 are formed so as to be laminated in this order on the outer peripheral surface 1b.

Both the buffer layer part L1, the reinforcing layer part L2, and the outer layer part L3 are formed in a tubular shape so as to be coaxial with the central axis O of the inner-layer tube 1.

The buffer layer part L1, the reinforcing layer part L2, and the outer layer part L3 may be configured such that three layers of different materials are closely adhered to each other in a laminated portion. However, in the present embodiment, as an example, an elastomer layer 2 made of a polymer elastomer is included in common in each layer.

The buffer layer part L1 is configured by arranging a cylindrical resin blade 3 (second mesh-like body) surrounding the outer peripheral surface 1b of the inner-layer tube 1 inside the elastomer layer 2.

The reinforcing layer part L2 is formed by arranging a cylindrical metal blade 4 (reinforcing member, first mesh-like body) surrounding the outer peripheral side of the resin blade 3 inside the elastomer layer 2.

In the buffer layer part L1 and the reinforcing layer part L2, the elastomer layer 2 penetrates in the layer thickness direction.

The outer layer part L3 is constituted by an elastomer layer 2 surrounding the outer peripheral side of the metal blade 4.

The outer peripheral surface 1b of the inner-layer tube 1 is in close contact with the inner peripheral portion of the resin blade 3 and the inner peripheral surface 2a of the elastomer layer 2.

The boundary between the buffer layer part L1 and the reinforcing layer part L2 is defined by the envelope surface where the resin blade 3 and the metal blade 4 contact each other. In the present embodiment, the layer thickness of the buffer layer part L is equal to the thickness of the resin blade 3. However, the buffer layer part L1 may include a layered portion consisting only of the elastomer layer 2 And formed on the inner side of the resin blade 3 (between the resin blade 3 and the inner-layer tube 1) and the outer side (between the resin blade 3 and the metal blade 4).

The boundary between the reinforcing layer part L2 and the outer layer part L3 is defined by the envelope surface on the outer peripheral side of the metal blade 4. In the present embodiment, the layer thickness of the reinforcing layer part L2 is equal to the thickness of the metal blade 4.

As the material of the elastomer layer 2, thermoplastic elastomer such as, for example, urethane type thermoplastic elastomer, ester type thermoplastic elastomer, amide type thermoplastic elastomer, styrene type thermoplastic elastomer, olefin type thermoplastic elastomer, fluorine type thermoplastic elastomer, vinyl chloride type thermoplastic elastomer, or the like may be used.

As the material of the elastomer layer 2, vulcanized rubber such as, for example, natural rubber, isoprene rubber, butadiene rubber, styrene-butadiene rubber, butyl rubber, ethylene-propylene rubber, chloroprene rubber, chlorosulfonated polyethylene rubber, nitrile rubber, silicone rubber, urethane rubber, acrylic rubber, fluorine rubber, or the like may be used.

As the elastomer layer 2 Different materials may be used for each portion of the channel tube. For example, the vulcanized rubber may be used for the curved portion of the channel tube, and the thermoplastic elastomer may be used for the other portions.

As the elastomer layer 2A porous body or a foam of the above-mentioned material or composite material may be used. In this case, the flexibility of the channel tube for endoscope 11 is improved.

Among the above-mentioned materials as the elastomer layer 2, particularly preferable materials are thermoplastic elastomers in which peroxide crosslinked rubber or peroxide crosslinked rubber is dispersed. For peroxide crosslinking, organic peroxide crosslinking is more preferred.

Specific examples of such particularly preferable materials include, for example, peroxide-crosslinked fluororubbers, polyurethane elastomers in which particles of silicone rubber are dispersed, and the like.

The peroxide-crosslinked rubber or the thermoplastic elastomer in which the peroxide-crosslinked rubber is dispersed is excellent in softness and hardly adheres to the below-mentioned resin blade 3 and metal blade 4, so that the stretchability of the buffer layer part L1 and the reinforcing layer part L2 is improved. This further improves the flexibility of the channel tube for endoscope 11.

The resin blade 3 is composed of a mesh-like body (a second mesh-like body) formed by an element wire (second element wire) made of resin or elastomer. The shape of the element wire is not particularly limited. Examples of the shape of the element wire include round wire, flat wire, twisted wire, temporary woven wire, and the like.

The element wire used for the resin blade 3 may be a single type of element wire, or a plurality of types of element wires different in at least one of material and shape may be combined. In the resin blade 3, when a plurality of types of strands are used, they may be twisted together or may be arranged differently from each other. When the disposition positions are different from each other, for example, the type of element wires extended along the longitudinal direction of the buffer layer section L1 may be different from the type of element wires circled in the circumferential direction.

In a case where the mesh-like body used as the resin blade 3 is knitted or woven with an element wire, knitting and weaving methods are not particularly limited. Examples of the manner of knitting or weaving the mesh-like body include plain weave, twill weave, satin weave, torsion lace, knot mesh, non-knot net, and the like.

Further, the resin blade 3 is not limited to a structure knitted or woven with a strand as long as it is a mesh-like body. For example, as the resin blade 3, a mesh-like body such as a punching mesh, a drawing net, or the like may be used.

The material of the resin blade 3 is not particularly limited as long as it is a resin material or a polymer elastomer material capable of forming a flexible mesh-like body.

In the case where the resin blade 3 is made of, for example, a resin material, one or more kinds of resin materials may be selected from the various general-purpose plastics, engineering plastics, super engineering plastics, and fluorocarbon resins exemplified as the material of the inner-layer tube 1.

In the case where the resin blade 3 is made of, for example, a polymer elastomer material, one or more kinds of polymer elastomer materials may be selected from the above-mentioned thermoplastic elastomers exemplified as the material of the inner-layer tube 1.

The type of material used for the resin blade 3 may be the same as or different from the type of the inner-layer tube 1.

Each of the above-described materials may be used alone for the resin blade 3, or may be used as a composite material in which a plurality of materials are combined.

As a material constituting the resin blade 3, a material excellent in toughness is more preferable. Examples of materials having particularly excellent toughness include polytetrafluoroethylene, tetrafluoroethylene-perfluoroalkylvinylether copolymer, polyamide, and the like.

The metal blade 4 is used for reinforcing the channel tube for endoscope 11.

The metal blade 4 is composed of a mesh-like body (first mesh-like body) formed by a metal element wire (first element wire). The shape of the element wire is not particularly limited. Examples of the shape of the element wire include a round wire, a flat wire, a twisted wire, and the like.

The metal element wire used for the metal blade 4 may be a single type of element wire, or a plurality of types of element wires different in at least one of material and shape may be combined. In the case where a plurality of kinds of element wires are used in the metal blade 4, they may be twisted together or may be arranged differently.

In the case where the mesh-like body used as the metal blade 4 is knitted or woven with an element wire, the way of knitting and weaving is not particularly limited. Examples of the manner of knitting or weaving the mesh-like body include, for example, plain weave, twill weave, satin weave, non-knot net, and the like.

Examples of the material of the metal strands constituting the metal blade 4 include, for example, copper, copper alloy, piano wire, stainless steel, titanium, titanium alloy, nickel titanium alloy, tungsten, tungsten alloy, nickel alloy, cobalt alloy, amorphous metal, and the like.

An example of a copper alloy is brass. An example of a titanium alloy is 64 titanium. An example of the tungsten alloy is a tungsten (W)-rhenium (Re) alloy. Examples of the nickel alloy include a nickel (Ni)-chromium (Cr)-iron (Fe) alloy and a nickel-chromium-iron-niobium (Nb)-molybdenum (Mo) alloy. An example of the cobalt alloy is a cobalt (Co)-chromium alloy.

As the material constituting the metal blade 4, it is more preferable that it is a metal which is excellent in toughness and hardly corroded by autoclave sterilization. An example of a metal which is excellent in toughness and is less susceptible to corrosion by autoclave sterilization is, for example, stainless steel.

In the channel tube for endoscope 11 having such a configuration, after the resin blade 3 and the metal blade 4 are laminated in this order around the outer peripheral surface 1b of the inner-layer tube 1, the elastomer layer 2 is formed to cover the metal blade 4. For forming the elastomer layer 2, for example, extrusion molding may be used. The elastomer layer 2 is brought into close contact with the outer peripheral surface 1b of the inner-layer tube 1 through a reticulated gap between the metal blade 4 and the resin blade 3.

Before laminating the resin blade 3 and the second lens frame 4 to the inner-layer tube 1, the outer peripheral surface 1b may be subjected to a surface treatment for improving the adhesion to the elastomer layer 2.

The inner peripheral surface 1a of the inner-layer tube 1 may be covered with a coating resin in advance.

The channel tube for endoscope 11 is reinforced by a reinforcing layer part L2 including a hard metal blade 4.

In the reinforcing layer part L2, a metal blade 4, which is a cylindrical mesh-like body formed of metal element wires, is buried in the elastomer layer 2. Since the inner peripheral surface 2a of the elastomer layer 2 is in close contact with the outer peripheral surface 1b of the inner-layer tube 1, for example, when the inner-layer tube 1 receives an external force of deforming, the metal blade 4 also receives an external force which is similarly deformed.

Since the metal blade 4 is a mesh-like body, it has flexibility by changing the shape of the mesh with deformation. Further, the metal blade 4 has stretchability in the direction along the center axis O of the inner-layer tube 1 by changing the shape of the mesh.

Since the metal blade 4 is formed of a harder metal element wire than the material of the inner-layer tube 1, it has a shape retaining property to hold a tubular shape against an external force. Since it is made of metal, it functions as a reinforcing member that suppresses deformation of the inner-layer tube 1 integrated via the elastomer layer 2. Therefore, for example, when an external force acting to crush the inner-layer tube 1 acts, or when the channel tube for endoscope 11 is bent, it becomes a member resisting collapse of the inner peripheral surface 1a of the inner-layer tube 1.

That is, according to the channel tube for endoscope 11, since the metal blade 4 has an excellent shape-retaining action, the kink resistance is further improved. In addition, since the mesh-like body woven with a hard wire easily expands and contracts in the axial direction O, the flexibility is further improved.

For example, as a comparative example, a channel tube for endoscope is considered in which the metal blade 4 is in close contact with the outer peripheral surface 1b of the inner-layer tube 1. In this case, the shape retention effect of the metal blade 4 to suppress deformation of the inner-layer tube 1 is enhanced. However, for example, when a channel tube for endoscope is curved, the metal blade 4 is strongly pressed against the inner-layer tube 1 at a portion subjected to large deformation. Since the inner peripheral surface 1a of the inner-layer tube 1 is deformed inward on the back side of the contact portion with the metal blade 4, unevenness due to deformation occurs on the inner peripheral surface 1a. When a rigid member such as a treatment tool inserted into the channel tube for endoscope 11 slides on this inner peripheral surface 1a, severe abrasion occurs at the convex portion of the inner peripheral surface 1a. As a result, kinks starting from abrasion are likely to occur.

In contrast to such a comparative example, in the case of the channel tube for endoscope 11 of the present embodiment, the buffer layer part L1 is disposed between the metal blade 4 and the inner-layer tube 1. Since the elastomer layer 2 And the resin blade 3 constituting the buffer layer part L1 are both softer than the metal blade 4, the buffer layer part L1 has a relatively soft layered portion compared to the reinforcing layer part L2. Further, the resin blade 3 prevents direct contact between the metal blade 4 and the inner-layer tube 1.

When the channel tube for endoscope 11 is bent, the stress generated in the channel tube for endoscope 11 is relaxed by the buffer layer part L1. That is, in the buffer layer part L1, stress relaxation effect can be obtained by deformation of the elastomer layer 2 And movement of the resin blade 3 relative to the elastomer layer 2 (hereinafter collectively referred to as deformation of the buffer layer part L1). The buffer layer part L has cushioning properties against compression by external force.

When a material having low adhesiveness to the strand of the resin blade 3 is selected as the material of the elastomer layer 2, the stress relaxation effect due to the relative displacement between the resin blade 3 and the elastomer layer 2 Becomes particularly high.

For example, the pressing force from the metal blade 4 toward the inner-layer tube 1 is dispersed in the buffer layer part L1 through deformation of the buffer layer part L1. The pressing force from the metal blade 4 is transmitted to the outer peripheral surface 1b of the inner-layer tube 1 while spreading beyond the contact portion with the metal blade 4 via the elastomer layer 2 And the resin blade 3. Therefore, since the pressing force applied to the outer peripheral surface 1b of the inner-layer tube 1 is also dispersed, the local deformation of the inner-layer tube 1 at the portion facing the metal blade 4 is reduced.

As a result, the inner peripheral surface 1a has a smooth shape conforming to the curved shape, so that even if it slides on a hard member such as a treatment tool, abrasion is locally reduced compared with the abrasion of the protrusion in the above-described comparative example.

Therefore, the occurrence of kink originating from the abrasion marks of the inner peripheral surface 1a decreases, and the kink resistance is improved.

For example, even when the channel tube 11 for endoscope is not curved, there is a case where a convex portion of a hard member such as a treatment tool inserted into the inner peripheral surface 1a presses the inner-layer tube 1. Also in this case, the pressing force from the hard member to the inner-layer tube 1 is dispersed by deformation of the buffer layer part L1 and is transmitted to the metal blade 4. The metal blade 4 and the inner-layer tube 1 are not brought into direct contact with each other. Therefore, as compared with the case where the metal blade 4 and the elastomer layer 2 Are in direct contact with each other, the reaction from the metal blade 4 is reduced, and the contact between the hard member and the inner-layer tube 1 is weakened. As a result, even if the hard member slides, abrasion due to sliding is reduced.

For example, the channel tube for endoscope 11 sometimes receives an external force as the outer peripheral surface 2b constituting the outermost peripheral portion comes into contact with another member or the like. In this case, the external force is transmitted to the inside via the outer layer part L3 and the metal blade 4. At this time, the metal blade 4 is sandwiched between the softer outer layer part L3 composed of the elastomer layer 2 And the buffer layer part L1. Therefore, the external force is alleviated through the outer layer part L3 and is transmitted to the metal blade 4 as a dispersed pressing force. Further, the pressing force transmitted by the metal blade 4 to the inside is dispersed and weakened in a wider range by the stress relaxation effect of the buffer layer part L1, and transmitted to the inner-layer tube 1. The amount of deformation on the inner peripheral surface 1a of the inner-layer tube 1 is remarkably reduced also in the concave deformation of the outer peripheral surface 2b due to the influence of the external force. As a result, local abrasion due to a hard member such as a treatment tool sliding on the inner peripheral surface 1a is reduced.

Therefore, the occurrence of kink originating from the abrasion marks of the inner peripheral surface 1a decreases, and the kink resistance is improved.

As described above, according to the channel tube for endoscope 11 of the present embodiment, even if it includes the metal blade 4, it is possible to improve the kink resistance by reducing the occurrence of kink starting from the abrasion marks of the inner peripheral surface 1a since the buffer layer part L1 is disposed between the metal blade 4 and the inner-layer tube 1.

### (First Modification)

A channel tube for endoscope according to a modified example (first modified example) of the present embodiment will be described.

FIG. 2 is a schematic cross-sectional view showing a configuration example of a channel tube for endoscope of a modified example (first modified example) of the first embodiment of the present invention.

As shown in FIG. 2, the channel tube for endoscope 11A of this modified example is different from the channel tube for endoscope 11 of the first embodiment in that instead of the buffer layer part L1 and the outer layer part L3, the buffer layer part L11 (inner buffer layer part) and an outer layer part L113 are provided.

Hereinafter, differences from the first embodiment will be mainly described.

The buffer layer part L11 is a layered portion made of a polymer elastomer that is softer than the reinforcing layer part L2.

As the material of the buffer layer part L1, one or more materials may be selected from thermoplastic elastomers and rubbers that can be used as the elastomer layer 2 in the first embodiment.

The material of the buffer layer part L11 may be the same as or different from the material of the elastomer layer 2 in this modification example. It is more preferable that the material of the buffer layer part L1l is selected to be softer than the material of the outer layer part L13 described later.

In the following, as an example, a case will be described in which the buffer layer part L11 is made of a material different from the later-described outer layer part L13.

The inner peripheral surface L11a of the buffer layer part L11 is in close contact with the outer peripheral surface 1b of the inner-layer tube 1.

The outer peripheral surface L11b of the buffer layer part L11 is in contact with the inner peripheral portion of the metal blade 4.

The outer layer part L13 is formed of an elastomer layer 2A made of the same material as the elastomer layer 2 in the first embodiment.

The elastomer layer 2A penetrates the metal blade 4 and is in close contact with the outer peripheral surface L11b of the buffer layer part L11. That is, the inner peripheral surface 2c of the elastomer layer 2A is in close contact with the outer peripheral surface L1b of the buffer layer part L11.

The elastomer layer 2A penetrating the metal blade 4 together with the metal blade 4 constitutes a reinforcing layer part L2.

In order to manufacture such a channel tube for endoscope 11A, for example, after forming the buffer layer part L11 on the outer peripheral surface 1b of the inner-layer tube 1 by extrusion molding, the metal blade 4 is disposed on the outer peripheral surface L11b and the elastomer layer 2A is formed by extrusion molding.

According to the channel tube for endoscope 11A, since the buffer layer part L11 is provided instead of the buffer layer part L1 in the first embodiment, as in the first embodiment, abrasion of the inner peripheral surface 1a of the channel tube for endoscope 11A is reduced and the kink resistance can be improved.

In particular, in this modified example, the buffer layer part L11 does not include a member such as the resin blade 3, so that it is easier to manufacture.

Furthermore, in this modified example, the airtightness and liquid tightness of the channel tube for endoscope 11A are further improved by the buffer layer part L11.

### Second Embodiment

A channel tube for endoscope according to a second embodiment of the present invention will be described.

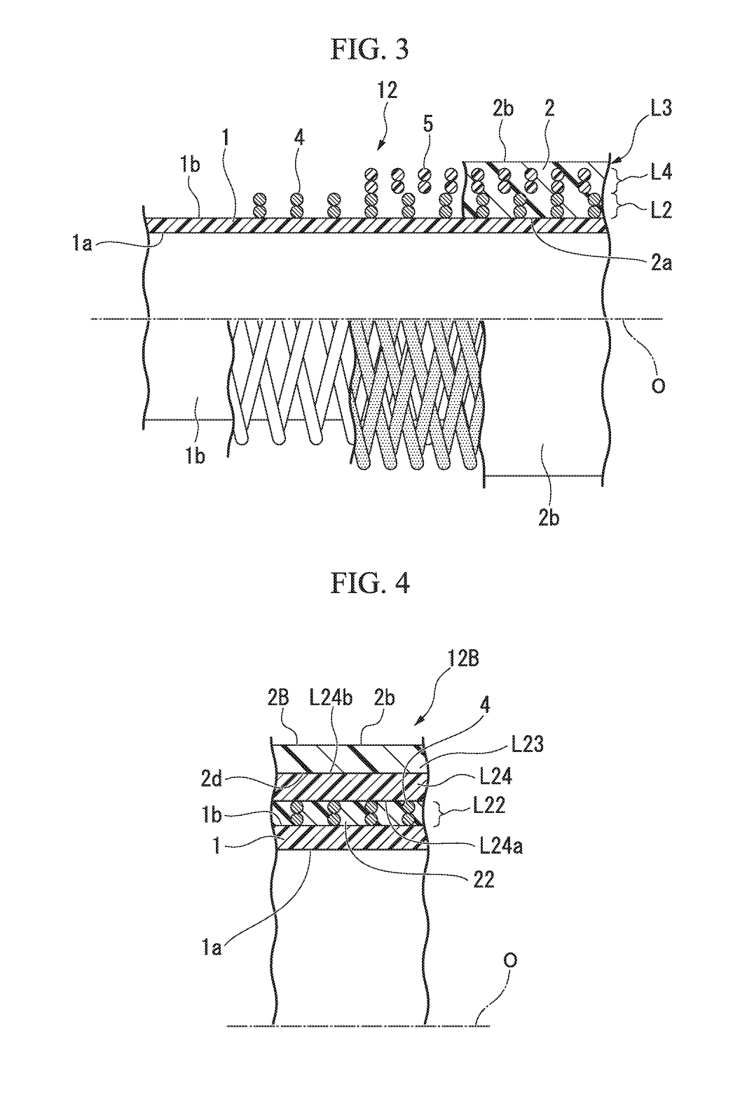

FIG. 3 is a schematic partial sectional view showing a configuration example of a channel tube for endoscope according to a second embodiment of the present invention.

As shown in FIG. 3, in the channel tube for endoscope 12 of the present embodiment, the buffer layer part L1 of the channel tube for endoscope 1 of the first embodiment is eliminated, and a buffer layer part L4 (outer buffer layer part) is additionally provided between the reinforcing layer part L2 and the outer layer part L3.

Hereinafter, differences from the first embodiment will be mainly described.

In the reinforcing layer part L2 in the present embodiment, the inner peripheral portion of the metal blade 4 is in contact with the outer peripheral surface 1b of the inner-layer tube 1. Therefore, the inner diameter of the metal blade 4 of this embodiment is changed in accordance with the outer diameter of the inner-layer tube 1.

The elastomer layer 2 penetrating the metal blade 4 is in close contact with the outer peripheral surface 1b.

The buffer layer part L4 is composed of a tubular resin blade 5 (second mesh-like body) surrounding the outer periphery of the reinforcing layer part L2 and an elastomer layer 2 penetrating the resin blade 5.

The resin blade 5 is formed of a mesh-like body configured similarly to the resin blade 3 in the first embodiment. However, the inner diameter of the resin blade 5 is aligned with the outer diameter of the reinforcing layer part L2.

The channel tube for endoscope 12 is formed by laminating and arranging the metal blade 4 and the resin blade 5 in this order on the inner-layer tube 1 and then forming the elastomer layer 2 By, for example, extrusion molding, whereby it is manufactured in the same way as the first embodiment.

In the channel tube for endoscope 12 having such a configuration, while the channel tube for endoscope 11 of the first embodiment has the buffer layer part L1 between the inner-layer tube 1 and the reinforcing layer part L2, the buffer layer part L4 having the same structure as that of the buffer layer part L1 is disposed between the reinforcing layer part L2 and the outer peripheral surface 2b of the elastomer layer 2, which is different to the first embodiment.

Like the buffer layer part L1 in the first embodiment, the buffer layer part L4 has cushioning properties against compression of external force, and therefore has a stress relaxation effect.

Therefore, similarly to the first embodiment, abrasion of the inner peripheral surface 1a can be reduced and kink resistance can be improved.

For example, when the inner-layer tube 1 is pressed against the metal blade 4 by an external force, in the present embodiment, the elastomer layer 2 and the metal blade 4 are in contact with each other, but the buffer layer part L4 is arranged outside the metal blade 4. Thereby, the metal blade 4 pressed outward from the inner-layer tube 1 can escape to the outer side by deforming the buffer layer part L4. As a result, since the pressing force between the metal blade 4 and the inner-layer tube 1 is reduced, local deformation of the inner peripheral surface 1a at the contact portion with the metal blade 4 is reduced.

For example, when the channel tube for endoscope 12 receives an external force from the outside through the outer layer part L3, external force is dispersed and transmitted to the metal blade 4 due to stress relaxation effect due to deformation of the buffer layer part L4, and the amount of deformation of the outer peripheral surface 2b is reduced. As a result, since the pressing force and the deformation amount transmitted to the inner-layer tube 1 via the metal blade 4 are reduced, the local deformation of the inner peripheral surface 1a at the contact portion with the metal blade 4 is reduced.

### (Second Modification)

A description will be given of a channel tube for endoscope of a modified example (second modified example) of the present embodiment.

FIG. 4 is a schematic cross-sectional view showing a configuration example of a channel tube for endoscope of a modified example (second modified example) of the second embodiment of the present invention.

As shown in FIG. 4, the channel tube for endoscope 12B of this modification is configured such that the outer layer part L3, the buffer layer part L4, and the reinforcing layer part L2 of the channel tube for endoscope 12 of the second embodiment are replaced by an outer layer part L23, a buffer layer part L24 (outer buffer layer part), and a reinforcing layer part L22.

Hereinafter, differences from the second embodiment will be mainly described.

The outer layer part L23 is formed of an elastomer layer 2B made of the same material as the elastomer layer 2 in the first embodiment.

The inner peripheral surface 2d of the elastomer layer 2B is in close contact with the outer peripheral surface L24b of the buffer layer part L24 described later.

The buffer layer part L24 is a layered portion made of a polymer elastomer that is softer than the reinforcing layer part L22 described later.

As the material of the buffer layer part L24, one or more materials may be selected from the thermoplastic elastomer and rubber that can be used as the elastomer layer 2 in the first embodiment.

The material of the buffer layer part L24 may be the same as or different from the material of the elastomer layer 2B in this modification example. It is more preferable that the material of the buffer layer part L24 is selected to be softer than the material of the outer layer part L23.

In the following, as an example, the case where the buffer layer part L24 is made of a material different from that of the outer layer part L23 will be described.

The outer peripheral surface L24b of the buffer layer part L24 is in close contact with the inner peripheral surface 2d of the outer layer part L23.

The inner peripheral surface L24a of the buffer layer part L24 is in contact with the outer peripheral portion of the metal blade 4 of the reinforcing layer part L22 described later.

The reinforcing layer part L22 includes an elastomer layer 22 made of a polymer elastomer and a metal blade 4 arranged inside the elastomer layer 22 and similar to the second embodiment described above.

The elastomer layer 22 is made of the same material as the elastomer layer 2 in the first embodiment. The material of the elastomer layer 22 may be the same as or different from that of the elastomer layer 2B in the present embodiment.

In order to manufacture such a channel tube for endoscope 12B, after arranging the metal blade 4 on the inner-layer tube 1, the elastomer layer 22 is formed by, for example, extrusion molding, whereby the reinforcing layer part L22 is formed. Thereafter, the buffer layer part L24 and the outer layer part L23 are formed in this order on the outer peripheral surface of the reinforcing layer part L22 by extrusion molding or the like.

According to the channel tube for endoscope 12B, since the buffer layer part L24 is provided instead of the buffer layer part L4 in the second embodiment, as in the second embodiment, it is possible to improve the kink resistance by reducing the abrasion of the inner peripheral surface 1a of the channel tube for endoscope 12B.

Particularly, in this modified example, since the buffer layer part L24 does not include a member such as the resin blade 5, it is easier to manufacture. Furthermore, in this modified example, the airtightness and liquid tightness of the channel tube for endoscope 12B are further improved by the buffer layer part L24.

### Third Embodiment

A channel tube for endoscope according to a third embodiment of the present invention will be described.

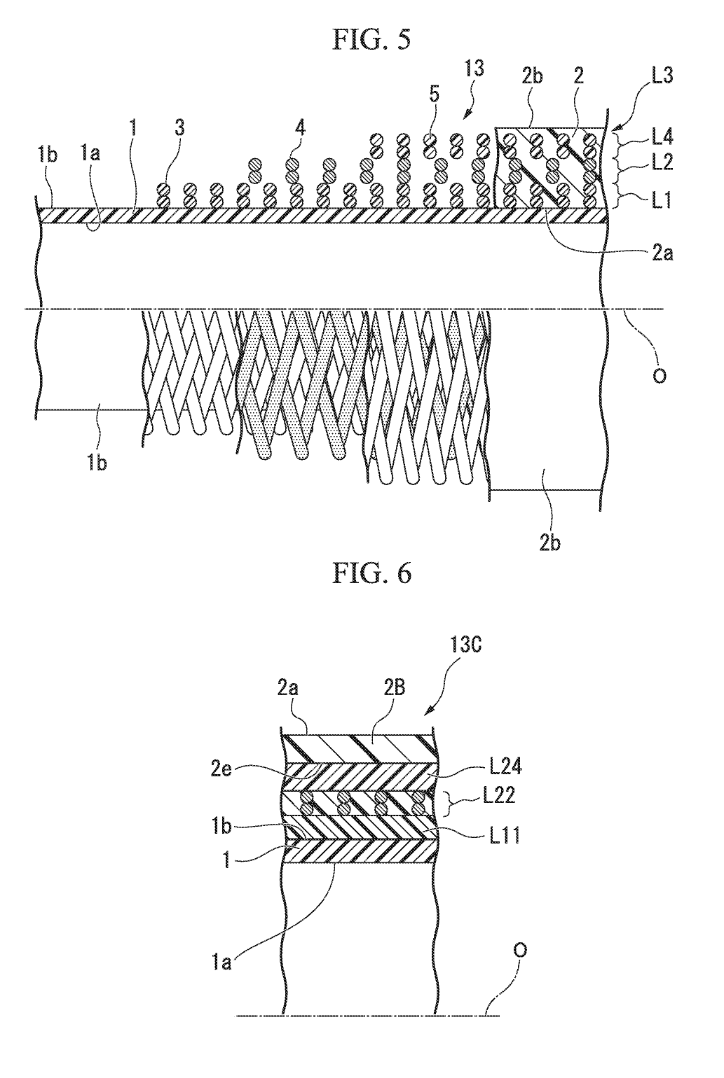

FIG. 5 is a schematic partial sectional view showing a configuration example of a channel tube for endoscope according to a third embodiment of the present invention.

As shown in FIG. 5, the channel tube 13 for endoscope according to the present embodiment is configured such that the buffer layer part L4 that is the same as that of the embodiment of FIG. 2 is additionally provided between the reinforcing layer part L2 and the outer layer part L3 of the channel tube for endoscope 11 of the first embodiment. However, the inner diameter of the resin blade 5 in the buffer layer part L4 is matched with the outer diameter of the metal blade 4 in the reinforcing layer part L2.

Hereinafter, differences from the first and second embodiments will be mainly described.

Since the reinforcing layer part L2 is sandwiched between the buffer layer parts L1 and L4, the channel tube for endoscope 13 of the present embodiment has both functions of the first embodiment and the second embodiment.

The buffer layer parts L and L4 are disposed inside and outside the reinforcing layer part L2 of the present embodiment. Therefore, the buffer layer parts L1 and L4 in the present embodiment more effectively relieve the external force from the inside and the external force from the outside, respectively. Therefore, as compared with the channel tube for endoscope 11 of the first embodiment and the channel tube for endoscope 12 of the second embodiment, abrasion of the inner peripheral surface 1a is further reduced, so that the kink resistance is further improved.

### Third Modified Example

A description will be given of a channel tube for endoscope of a modified example (third modified example) of the present embodiment.

FIG. 6 is a schematic cross-sectional view showing a configuration example of a channel tube for endoscope of a modified example (third modified example) of the third embodiment of the present invention.

As shown in FIG. 6, the channel tube 13C for endoscope according to the present modification is configured such that the buffer layer part L11 of the channel tube for endoscope 11A according to the first modification of the first embodiment is formed between the inner-layer tube 1 of the channel tube for endoscope 12B and the reinforcing layer part L22 of the second modification of the second embodiment.

Hereinafter, differences from the first modified example and the second modified example will be mainly described.

The channel tube for endoscope 13C according to the present modification has both functions of the first modified example and the second modified example since the reinforcing layer part L22 is sandwiched between the buffer layer parts L11 and L24.

The buffer layer parts L11 and L24 are disposed on the inner side and the outer side of the reinforcing layer part L22 of the present modification, respectively. Therefore, the external force from the inside and the external force from the outside are more effectively relieved by the buffer layer parts L11 and L24 in the present modification. Therefore, as compared with the channel tube for endoscope 11A of the first modified example and the channel tube for endoscope 12B of the second modified example, abrasion of the inner peripheral surface 1a is further reduced, so that the kink resistance is further improved.

### Fourth Embodiment

A channel tube for endoscope according to a fourth embodiment of the present invention will be described.

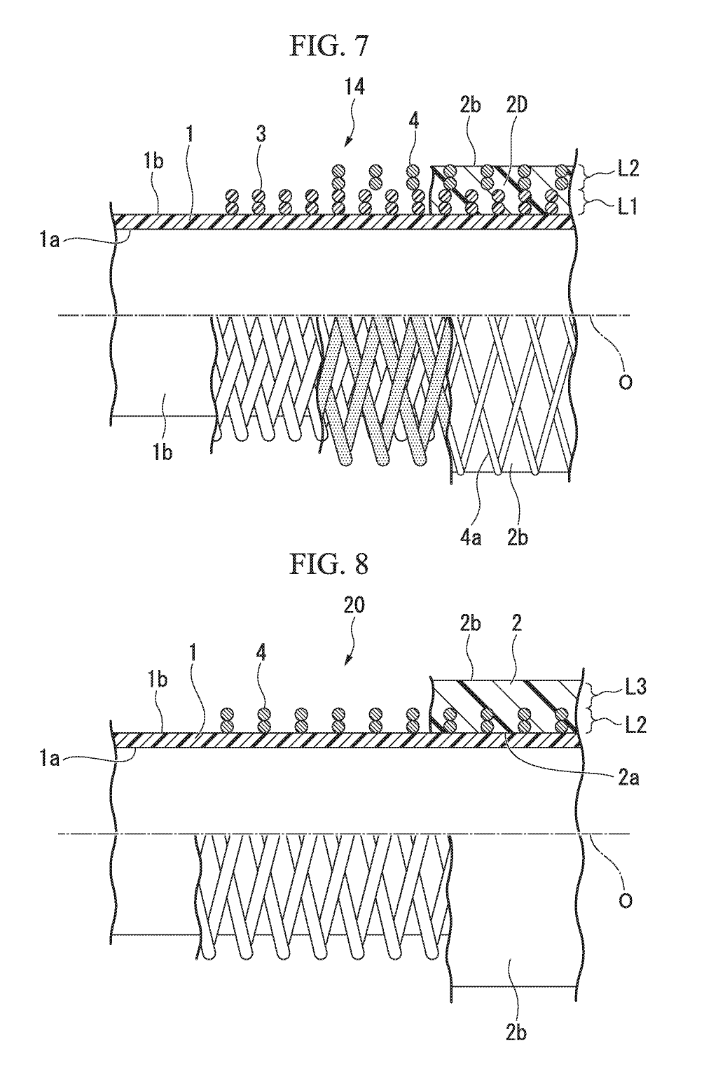

FIG. 7 is a schematic partial sectional view showing a configuration example of a channel tube for endoscope according to a fourth embodiment of the present invention.

As shown in FIG. 7, the channel tube for endoscope 14 of the present embodiment is configured such that the outer layer part L3 of the channel tube for endoscope 11 of the first embodiment is eliminated and an elastomer layer 2D is provided instead of the elastomer layer 2.

Hereinafter, differences from the first embodiment will be mainly described.

The elastomer layer 2D is different in layer thickness from the elastomer layer 2 of the first embodiment. The elastomer layer 2D is configured such that a part of the metal blade 4 is exposed to the outside from the outer peripheral surface 2b as an exposed portion 4a.

Further, in the elastomer layer 2D, a material having low adhesion with respect to the metal blade 4 and the resin blade 3 is used.

The channel tube for endoscope 13 of the present embodiment has the same function as the first embodiment by the same buffer layer part L1 as in the first embodiment. Therefore, as in the first embodiment, abrasion of the inner peripheral surface 1a is further reduced, so that the kink resistance is improved.

Furthermore, in this embodiment, since the buffer layer part L4 is exposed on the outer peripheral surface 2b of the elastomer layer 2D, the contact portion between the metal blade 4 and the resin blade 3 is exposed to the outside along the exposed portion 4a. In this embodiment, as the material of the elastomer layer 2D, a material having low adhesiveness to the metal blade 4 and the resin blade 3 is selected. Thereby, in the channel tube for endoscope 14, for example, when the layer elastomer 2 is deformed due to bending when used, the elastomer layer 2D and the metal blade 4/the resin blade 3 slide relative to each other and are relatively displaced. Therefore, an interface that is relatively movable and separable is formed between the elastomer layer 2D and the metal blade 4/the resin blade 3. As a result, in the interior of the elastomer layer 2D, a minute gap penetrating in the thickness direction of the elastomer layer 2D is formed, and the airtightness and liquid tightness of the elastomer layer 2 are lowered.

Therefore, when cracks penetrate in the thickness direction of the inner-layer tube 1 of the channel tube for endoscope 14 or damaged scratches penetrate, the airtightness and liquid tightness of the channel tube for endoscope 14 is lost.

Therefore, in the channel tube for endoscope 14, it is easy to inspect the perforation of the inner-layer tube 1, and the perforation detection is excellent. For example, when compressed air is fed into the through hole of the channel tube for endoscope 14, it can be detected that the hole is opened in the inner-layer tube 1 by air leakage.

As a result, for example, the elastomer layer 2 is air-tight, despite being a defective product having a hole in the inner-layer tube 1, so that it will not pass the perforation inspection. Therefore, there is no possibility that the hole of the inner-layer tube 1 will be overlooked by the hole opening inspection.

In the description of each of the above embodiments and modifications, the metal blade is used as the reinforcement member of the reinforcement layer part, but the reinforcement member is not limited to the metal blade.

For example, as the reinforcing member, a resin blade harder than the resin blade included in the buffer layer part may be used.

For example, as a reinforcing member, a coil made of a metal or a hard resin may be used.

In the description of each of the above embodiments and modifications, the reinforcement layer part is formed of a polymer elastomer and a metal blade, but the reinforcement layer part may further include a reinforcement member other than the metal blade.

For example, provisional weaving yarns, metal coils, or the like formed of highly stretchable wires oriented in the axial direction, for example, polyurethane, polyester, polyamide, fluororesin, or the like may be arranged in the reinforcing layer part.

### Example

Examples 1 to 8 of a channel tube for endoscope corresponding to the above-described first embodiment, each modification example, and fourth embodiment will be described together with comparative example 1. The schematic configuration of each example and comparative example is shown in the following Table 1.

TABLE 1

| | | | BUFFER LAYER PART | | |
|---|---|---|---|---|---|
| | INNER LAYER TUBE | | | MESH-LIKE | |
| | | SURFACE | LAYER | BODY | |
| | MATERIAL | TREATMENT | MATERIAL | MATERIAL | POSITION |
| EXAMPLE 1 | POLY-ETHYLENE | NO | FLUORO-RUBBER | — | INSIDE |
| | RESIN | | | | |
| EXAMPLE 2 | POLY-ETHYLENE | NO | FLUORO-RUBBER | — | OUTSIDE |
| | RESIN | | | | |
| EXAMPLE 3 | POLY-ETHYLENE | NO | FLUORO-RUBBER | — | INSIDE/ |
| | RESIN | | | | OUTSIDE |
| EXAMPLE 4 | POLY-ETHYLENE | NO | POLY-URETHANE | PFA | INSIDE |
| | RESIN | | RESIN | WIRE | |
| EXAMPLE 5 | POLY-ETHYLENE | NO | FLUORO-RUBBER | — | INSIDE |
| | RESIN | | | | |
| EXAMPLE 6 | FLUORO-RESIN | YES | FLUORO-RUBBER | — | INSIDE |
| EXAMPLE 7 | POLY-ETHYLENE | NO | FLUORO-RUBBER | — | INSIDE |
| | RESIN | | | | |
| EXAMPLE 8 | POLY-ETHYLENE | NO | FLUORO-RUBBER | PFA | INSIDE |
| | RESIN | | | WIRE | |
| COMPARATIVE | POLY-ETHYLENE | NO | — | — | — |
| EXAMPLE | RESIN | | | | |

| | REINFORCEMENT LAYER | | OUTER LAYER | |
|---|---|---|---|---|
| | | MESH-LIKE | | EXPOSURE |
| | LAYER | BODY | | OF MESH- |
| | MATERIAL | MATERIAL | MATERIAL | LIKE BODY |
| EXAMPLE 1 | POLY-URETHANE | COPPER | POLY-URETHANE | NO |
| | RESIN | WIRE | RESIN | |
| EXAMPLE 2 | POLY-URETHANE | COPPER | POLY-URETHANE | NO |
| | RESIN | WIRE | RESIN | |
| EXAMPLE 3 | POLY-URETHANE | COPPER | POLY-URETHANE | NO |
| | RESIN | WIRE | RESIN | |
| EXAMPLE 4 | POLY-URETHANE | COPPER | POLY-URETHANE | NO |
| | RESIN | WIRE | RESIN | |
| EXAMPLE 5 | POLY-URETHANE | SUS | POLY-URETHANE | NO |
| | RESIN | WIRE | RESIN | |
| EXAMPLE 6 | POLY-URETHANE | COPPER | POLY-URETHANE | NO |
| | RESIN | WIRE | RESIN | |
| EXAMPLE 7 | FLUORO-RUBBER | COPPER | FLUORO-RESIN | NO |
| | | WIRE | | |
| EXAMPLE 8 | POLY-URETHANE | COPPER | POLY-URETHANE | YES |
| | RESIN | WIRE | RESIN | |
| COMPARATIVE | POLY-URETHANE | COPPER | POLY-URETHANE | NO |
| EXAMPLE | RESIN | WIRE | RESIN | |

### Example 1

Embodiment 1 is an embodiment of the channel tube for endoscope 11A (see FIG. 2) of the first modification of the first embodiment.

As shown in Table 1, polyethylene resin was used as the material of the inner-layer tube 1 (the reference numerals are omitted in Table 1, the same applies below). No surface treatment was applied to the inner-layer tube 1 of Example 1. The inner-layer tube 1 of Example 1 had an inner diameter of 3.2 mm and a wall thickness of 0.15 mm.

As the buffer layer part L11, a fluorine rubber having a layer thickness of 0.1 mm was used.

The metal blade 4 which is the first mesh-like body (“mesh-like body” in Table 1) used as a reinforcing member was formed by plain weaving copper wire having a diameter of 0.1 mm. The condition of knitting of the first mesh-like body was set to 1, the number of strikes was 16, 30 PPI.

As the elastomer layer 2A, a polyurethane resin having a Shore hardness of 65A was used. The layer thickness of the elastomer layer 2A was set to 0.5 mm. As a result, the elastomer layer 2A completely covered the metal blade 4, and the metal blade 4 was never exposed to the outside.

The channel tube for endoscope 11A of Example 1 as described above was manufactured as follows. First, fluorine rubber having a thickness of 0.1 mm was laminated on the outer peripheral portion of the inner-layer tube 1 by extrusion molding. Thereafter, in a state where the plain-weaved metal blade 4 was disposed, it was covered with a polyurethane resin so as to have a layer thickness of 0.5 mm by extrusion molding.

### Example 2

Example 2 is an example of the channel tube for endoscope 12B (see FIG. 4) of the second modification of the first embodiment.

The second embodiment is different from the first embodiment in that the positional relationship between the fluororubber and the metal blade 4 is opposite. Therefore, in Example 2, on the inner-layer tube 1, a reinforcing layer part L22 made of the same polyurethane resin and metal blade 4 as the elastomer layer 2A of Example 1 and a buffer layer part L24 made of the same fluororubber as in Example 1 were stacked in this order. In this embodiment, the buffer layer part L24 is disposed outside the reinforcing layer part L22.

### Example 3

Embodiment 3 is an embodiment of the channel tube for endoscope 13C (see FIG. 6) of the third modification of the first embodiment.

In Example 3, the buffer layer part L11 of Example 1 was disposed between the inner-layer tube 1 and the reinforcing layer part L22 of Example 2 described above. In the present embodiment, the buffer layer part L11 is disposed inside the reinforcing layer part L22, and the buffer layer part L24 is disposed on the outside, respectively.

### Example 4

Embodiment 4 is an embodiment of the channel tube for endoscope 11 (see FIG. 1) of the first embodiment.

In Example 4, instead of the fluororubber of the buffer layer part L11 of Example 1, the buffer layer part L1 composed of the polyurethane resin of the elastomer layer 2A of Example 1 and a resin blade 3 that is the second mesh-like body (“mesh-like body” in Table 1) is used. The layer thickness of the buffer layer part L1 was 0.1 mm.

The resin blade 3 of this modified example was formed by plain weaving that weaves a wire of a tetrafluoroethylene-perfluoroalkyl vinyl ether copolymer having a diameter of 0.05 mm (“PFA wire” in Table 1). The condition of knitting of the second mesh-like body was set to 1, the number of beating was 16, and 30 PPI.

### Example 5

The fifth embodiment is an embodiment of the channel tube for endoscope 11A of the first modification of the first embodiment.

Example 5 is an example in which the copper wire of the metal blade 4 of Example 1 was changed to a stainless steel wire rod (“SUS wire” in Table 1).

The metal blade 4 of this modified example was formed by plain weaving that waves SUS 304 WPB having a diameter of 0.1 mm. The condition of knitting of the mesh-like body was set to 1, the number of strikes was 16, and 30 PPI.

### Example 6

The sixth embodiment is an embodiment of the channel tube for endoscope 11A according to the first modification of the first embodiment.

Example 6 is an example in which the material of the inner-layer tube 1 of Example 1 is changed to fluororesin and the outer peripheral surface 1b of the inner-layer tube 1 is subjected to surface treatment.

As the material of the inner-layer tube 1 of this example, nonporous polytetrafluoroethylene was used. The shape of the inner-layer tube 1 was the same as in Example 1.

The outer peripheral surface 1b of the inner-layer tube 1 of this modified example was etched with a metal sodium solution.

### Example 7

The seventh embodiment is an embodiment of the channel tube for endoscope 11A of the first modification of the first embodiment.

Example 7 is an example in which a fluororubber is used as the elastomer layer 2A of Example 1 described above.

As the material of the elastomer layer 2A of this example, peroxide-crosslinked fluororubber was used. This fluororubber was formed to have a layer thickness of 0.5 mm on the outer peripheral surface 1b of the inner-layer tube 1 by extrusion molding in the same manner as in Example 1 described above. Thereby, the elastomer layer 2A completely covered the metal blade 4, and the metal blade 4 was never exposed to the outside.

### Example 8

The eighth embodiment is an embodiment of the channel tube for endoscope 14 (see FIG. 7) of the fourth embodiment.

In Example 8, the layer thickness of the elastomer layer 2A of Example 1 was changed to 0.3 mm, and the buffer layer part L11 was changed to the same buffer layer part L1 as in Example 4.

As a material of the elastomer layer 2D, peroxide-crosslinked fluororubber was used. This fluororubber was formed on the outer peripheral surface 1b of the inner-layer tube 1 by extrusion so that the layer thickness became 0.3 mm. As a result, a part of the metal blade 4 was exposed on the outer peripheral surface 2b of the elastomer layer 2D.

### Comparative Example 1

FIG. 8 is a schematic partial sectional view showing the configuration of the channel tube for endoscope of Comparative Example 1.

As shown in FIG. 8, Comparative Example 1 is an example in which the resin blade 5 of the channel tube for endoscope 12B (see FIG. 4) of the second embodiment is eliminated. The portion occupied by the resin blade 5 is occupied by the elastomer layer 2 of the outer layer part L3. Therefore, in Comparative Example 1, the reinforcing layer part L2 was laminated on the inner-layer tube 1, and the outer layer part L3 was laminated on the outer side thereof.

The layer thickness of the elastomer layer 2 in Comparative Example 1 was set to 0.5 mm as in Example 1 described above. On the inner-layer tube 1, the buffer layer part L1 and the outer layer part L3 were laminated. Thereby, the elastomer layer 2 completely covered the metal blade 4, and the metal blade 4 was never exposed to the outside.

### (Evaluation Method)

Evaluation of washing and disinfecting properties, kink resistance, flexibility, and perforation detection storage stability were performed using the channel tubes for endoscopes of Examples 1 to 8 and Comparative Example 1.

Evaluation items and respective evaluation results are shown in the following Table 2.

TABLE 2

| | EVALUATION RESULTS | | | | |
|---|---|---|---|---|---|
| | KINK RESISTANCE | | | | |
| | OF ABRASION | KINK | | PERFORATION | COMPREHENSIVE |
| | PORTION | RESISTANCE | FLEXIBILITY | DETECTABILITY | EVALUATION |
| EXAMPLE 1 | ◯ | ◯ | ◯ | X | ◯ |
| EXAMPLE 2 | ◯ | ◯ | ◯ | X | ◯ |
| EXAMPLE 3 | ⊚ | ◯ | ◯ | X | ◯ |
| EXAMPLE 4 | ◯ | ◯ | ⊚ | X | ◯ |
| EXAMPLE 5 | ◯ | ⊚ | ◯ | X | ◯ |
| EXAMPLE 6 | ⊚ | ◯ | ◯ | X | ◯ |
| EXAMPLE 7 | ◯ | ◯ | ⊚ | X | ◯ |
| EXAMPLE 8 | ◯ | ◯ | ⊚ | ◯ | ◯ |
| COMPARATIVE | X | ◯ | ◯ | X | X |
| EXAMPLE | | | | | |

### (Kink Resistance of Abrasion Portion)

The kink resistance of the abrasion portion of the inner-layer tube due to insertion and removal of a treatment tool such as a forceps can be said to be better as the amount of abrasion on the surface of the inner-layer tube is smaller. Therefore, after the test of repeatedly inserting and removing the forceps was performed on the test sample of the channel tube for the endoscope, the abrasion portion was repeatedly bent, and the kink resistance of the abrasion portion was evaluated.

The sample to be tested was held in a curved state along a semicircle having a radius R. From the outer surface of this curved channel tube for endoscope, the upper surface of a cylinder with a diameter of 1.6 mm was pressed with a force of 2N toward the central axis of the channel tube.

In this state, biopsy forceps FB-25K (trade name; manufactured by Olympus Corporation) was repeatedly inserted and removed at a rate of 30 mm/sec inside each test sample.

The number of insertion and withdrawal was set to 1000 times for each test sample assuming that one reciprocation of the biopsy forceps was once.

After 1000 cycles of insertion and removal, the sample to be tested was grasped at two places with a distance of 250 mm in the longitudinal direction so that the abrasion portion was at the center. At this time, a tension of 1.96 N (200 gf) was applied to the test sample between the gripping positions.

Further, a pair of rollers with a radius of 9 mm with a distance of 5 mm interposed between the test samples was placed at the center position equally dividing the gripping position.

One of the gripping positions was fixed and the other one of the gripping positions was repeatedly rotated at 0°±90° with the center position being the center position and the test sample being in a straight state of 0°. As a result, the sample to be tested was repeatedly bent in two directions with each roller as a curved surface.

The turning +90°, returning to 0°, turning to −90°, returning to 0° is one time, and this flexing test is performed at a speed of 29 times/min, 1000 times for each test sample.

After the bending test was completed, the inner diameter of the bent portion was measured with a ball gauge.

Evaluation criteria was set as very good (“⊚” (very good) in Table 2) when the street diameter of the ball gauge was 3.2 or more, good (“◯” (good) in Table 2) when it was 3.18 or more but less than 3.2, defective (“X” (no good) in Table 2) when it was less than 3.18.

### (Kink Resistance)

The sample to be tested was grasped at two places with a distance of 250 mm in the longitudinal direction. At this time, a tension of 1.96 N (200 gf) was applied to the test sample between the gripping positions.

Further, a pair of rollers with a radius of 9 mm with a distance of 5 mm interposed between the test samples was placed at the center position equally dividing the gripping position.

One of the gripping positions was fixed and the other one of the gripping positions was repeatedly rotated at 0°±90° with the center position being the center position and the test sample being in a straight state of 0°. As a result, the sample to be tested was repeatedly bent in two directions with each roller as a curved surface.

The turning +90°, returning to 0°, turning to −90°, returning to 0° is one time, and this flexing test is repeated 5000 times for each test sample at a speed of 29 times/min.

After the bending test was completed, the inner diameter of the bent portion was measured with a ball gauge.

Evaluation criteria was set as very good (“⊚” (very good) in Table 2) when the street diameter of the ball gauge was 3.2 or more, good (“◯” (good) in Table 2) when it was 3.18 or more but less than 3.2, defective (“X” (no good) in Table 2) when it was less than 3.18.

### (Flexibility)

Flexibility was evaluated with the pushing force required to bend the test sample with three point bending.

In order to form both end fulcrums, two pulleys with a radius of 5 mm were arranged at 100 mm intervals and at equal heights in a vertical position. A test sample was placed on these pulleys. A push pull gauge was brought into contact with the portion located between the two pulleys from above. A pulley having a radius of 5 mm is provided at the contact portion of the push-pull gauge. The push pull gauge was measured at a speed of 20 mm/sec with a downward stroke of 40 mm, and the peak value of the pushing force amount when pushed in was measured.

Evaluation criteria was set as very good (“⊚” (very good) in Table 2) when the peak value of indentation force amount was less than 0.7 N, good (“◯” (good) in Table 2) when it was 0.7 N or more and less than 0.8 N, defective (“X” (no good) in Table 2) when it was 0.8 N or more.

### (Hole Opening Detectability)

A biopsy forceps used for evaluating washing and disinfecting properties was repeatedly inserted and removed to prepare a test sample having a hole in the inner-layer tube.

Compressed air with a gauge pressure of 0.1 MPa was fed from the other end with one end of the channel tube for endoscope closed, and air leakage from the outer surface of the channel tube was observed in the water.

Evaluation criteria was set as good (“◯” (good) in Table 2) when air leaks from the outer surface of perforated sample, defective (“X” (no good) in Table 2) when air did not leak.

### (Evaluation Results)

As shown in Table 1, in Examples 1 to 8, the kink resistance, the kink resistance and the flexibility of the abrasion portion were both “◯” or “⊚”, so the overall evaluation was good (“◯” (good) in Table 2).

On the other hand, in Comparative Example 1, since the kink resistance of the abrasion portion was poor, the overall evaluation was set to be defective (“X” (no good) in Table 2).

In particular, Example 3 was superior in the kink resistance of the abrasion portion. On the other hand, in Comparative Example 1, since there is no buffer layer part, the kink resistance of the abrasion portion was poor.

Regarding the flexibility, Examples 4 and 8 in which the buffer layer part was provided with a PFA wire net and Example 7 in which the elastomer layer was made of fluororubber were superior to Example 1. Example 7 does not have a mesh-like body in the buffer layer part, but since the fluorine rubber in the reinforcing layer part is hardly fixed to the mesh-like body made of copper wire, it is thought that the flexibility is improved as compared with Example 1.

Example 5 having the metal blade 4 with the plain weave of the SUS wire as the reinforcing layer part was further excellent in kink resistance as compared with Example 1.

Example 6, in which the inner-layer tube was made of nonporous polytetrafluoroethylene, was more excellent in kink resistance of the abrasion area than in Example 1.

In Example 8, in addition to good flexibility, good hole opening detectability was obtained. For this reason, it is understood that a configuration in which a part of the metal blade 4 is exposed on the outer peripheral surface as in the eighth embodiment is suitable especially when the hole opening detection property is required.

While each preferred embodiment and each modified example of the present invention has been described in conjunction with each of the embodiments, the present invention is not limited to each of these embodiments, each modification, and each embodiment. Additions, omissions, substitutions, and other changes in the configuration are possible without departing from the spirit of the present invention.

Also, the invention is not limited by the foregoing description, but only by the scope of the appended claims.

The present invention can be widely applied to a channel tube for endoscope, and it is possible to improve kink resistance by reducing abrasion of the inner-layer tube.

What is claimed is:

1. A channel tube for endoscope comprising:
an inner-layer tube inside which a through hole extending in a longitudinal direction is formed, the inner-layer tube having an elastomer or a flexible resin as a base material thereof;
an elastomer layer including a polymer elastomer, the elastomer layer being disposed so as to cover an outside of the inner-layer tube, and the surface of the elastomer layer being exposed to an outside;
a reinforcing layer part including a flexible reinforcing member, the reinforcing layer part being disposed so as to surround the inner-layer tube; and
a buffer layer part that deforms more readily than the reinforcing layer part, the buffer layer part being layered on the reinforcing layer part and disposed between the inner-layer tube and an outer peripheral surface of the elastomer layer.

2. The channel tube for endoscope according to claim 1, wherein the reinforcing member includes a first mesh-like body formed of a first element wire.

3. The channel tube for endoscope according to claim 2, wherein the buffer layer part includes a second mesh-like body formed of a second element wire that is softer than the first element wire.

4. The channel tube for endoscope according to claim 1, wherein the buffer layer part is disposed between the inner-layer tube and the reinforcing layer part.

5. The channel tube for endoscope according to claim 1, wherein the buffer layer part is disposed between the reinforcing layer part and an outer peripheral surface of the elastomer layer.

6. The channel tube for endoscope according to claim 1, wherein the buffer layer part includes:
an inner buffer layer part disposed between the inner-layer tube and the reinforcing layer part; and
an outer buffer layer part disposed between the reinforcing layer part and an outer peripheral surface of the elastomer layer.

7. The channel tube for endoscope according to claim 1, wherein the inner-layer tube is made of a fluororesin.

8. The channel tube for endoscope according to claim 3, wherein the elastomer layer is disposed so as to penetrate through the reinforcing layer part and the buffer layer part so as to be in close contact with an outer peripheral surface of the inner-layer tube, and the elastomer layer is formed of an elastomer having a lower adhesion to the first mesh-like body and the second mesh-like body than an adhesion to the inner-layer tube.

9. The channel tube for endoscope according to claim 8, wherein the elastomer includes an organic peroxide crosslinked rubber or a thermoplastic elastomer in which the organic peroxide crosslinked rubber is dispersed.

10. The channel tube for endoscope according to claim 3, wherein at least one of the first mesh-like body and the second mesh-like body is partly exposed to an outside from the outer peripheral surface.
